(19) United States
(12) Patent Application Publication
(10) Pub. No.: US 2019/0046620 A1
(43) Pub. Date: Feb. 14, 2019

(54) THERAPEUTIC INDUCTION OF TOLERANCE USING RECOMBINANT CELL SURFACE ANTIGENS

(71) Applicant: BLOODWORKS, SEATTLE, WA (US)

(72) Inventor: James Charles Zimring, Seattle, WA (US)

(73) Assignee: BLOODWORKS, SEATTLE, WA (US)

(21) Appl. No.: 16/077,386

(22) PCT Filed: Feb. 13, 2017
(86) PCT No.: PCT/US17/17706

§ 371 Date: Aug. 10, 2018

Related U.S. Application Data
(60) Provisional application No. 62/294,801, filed on Feb. 12, 2016.

(51) Int. Cl. A61K 39/00; C07K 14/74; C07K 14/705; C07K 14/54; C07K 14/495; A61K 47/60; A61P 37/06

(52) U.S. Cl. CPC A61K 39/0008; C07K 14/70539; C07K 14/70521; C07K 14/5428; C07K 14/495; C07K 2319/55; C07K 14/70575; A61P 37/06; A61K 2039/545; C07K 2319/30; A61K 47/60

(57) ABSTRACT

Compositions and methods for the induction of tolerance using recombinant cell surface antigens in a vertebrate subject are provided.

### FIELD

The invention relates to compositions and methods for the therapeutic induction of tolerance using recombinant cell surface antigens. The methods of the invention are useful for inducing tolerance to a variety of clinically relevant antigens, such as red blood cell or platelet antigens, or antigens involved in alloimmune or autoimmune diseases.

### INTRODUCTION

The ultimate goal of immuno-pharmacology is the regulation of the immune system in an antigen-specific fashion. This has long been accomplished in the context of inducing immunity, as vaccines that induce specific responses to pathogens have changed the landscape of infectious disease and have even been used to eradicate certain pathogens. However, inhibiting immune responses in an antigen-specific fashion, without down modulating general protective immunity has remained more elusive. General immunosuppression is still the approach used to treat autoimmunity and most organ transplant rejection. However, there are two exceptions where antigen-specific down modulation of immune responses has been achieved. The first is the use of RhoGam to prevent RhD—mothers from generating anti-D alloantibodies that can lead to fetal demise in utero. The second is the use of high dose factor VIII (fVIII) intravenously in order to shut off an anti-fVIII antibody inhibitor response in patients with hemophilia A who have become alloimmunized to replacement fVIII infusion. This high dose fVIII approach accomplishes antigen-specific down modulation of a pre-existing alloantibody.

The application of such approaches more widely would be highly desirable. The present invention provides additional methods for therapeutic induction of tolerance which is generally applicable to many clinically relevant conditions.

### SUMMARY

Described herein are compositions and methods for the therapeutic induction of tolerance using recombinant cell surface antigens.

In one aspect, the present invention provides a method for inducing immune tolerance against a cell surface antigen in a subject comprising administering to the subject an effective amount of a soluble n of the cell surface antigen sufficient to induce tolerance to said antigen in the subject.

In one embodiment of this aspect, the subject has previously been sensitized. In another embodiment, the subject has developed an antibody as a result of previous exposure to the antigen.

In various embodiments, the cell surface antigen is a red blood cell antigen. For example, the red blood cell antigen is an antigen within the Kell system comprising an allelic variant of the extracellular domain of the Kell glycoprotein. In some embodiments, the allelic variants are selected from the group consisting of K, k, Kpb, Kpa, Jsb and Jsa antigens.

In further embodiments, the red blood cell antigen is a variant selected from within the Duffy, Kidd, or Lewis systems. In other embodiments, the cell surface antigen is a platelet antigen, such as Human Platelet Antigens 1-16 (HPA 1-16).

In other embodiments, the antigen is a neutrophil antigen (HNA antigens). In yet further embodiments, the antigen is an HLA variant of MHC I or MHC II.

In some embodiments, the administration is performed prior or subsequent to the onset of a disease or condition that is caused by said cell surface antigen. In such embodiments, the disease or condition can be hemolytic transfusion reaction (HTR). Other diseases or conditions include Neonatal Alloimmune Immunethrombocytopenia (NAIT), Autoimmune Hemolytic Anemia, or Immune Thrombocytopenia (ITP). Yet further diseases or conditions include Rasmussen's encephylitis, Hashimotos thyroiditis, Grave's Disease, Pemphigus Vulgaris, Stiffs an syndrome, or Myasthenia gravis.

In various embodiments, the soluble form of the cell surface antigen is fused to the Fc domain of an immunoglobulin. In other embodiments, the soluble form of the cell surface antigen is fused to an immunomodulatory protein. Examples of immunomodulatory proteins include FasL, CTLA4, or an immunoregulatory cytokine, such as IL-10 or TGF-beta.

In further embodiments, the soluble antigen is modified with PEG, and in others, the soluble antigen is conjugated to a toxin.

### BRIEF DESCRIPTION OF THE FIGURES

FIG. 1 provides a diagram of results obtained from experiments conducted directed to blocking antibody-mediated RBC clearance by non-hemolytic antibodies.

### DETAILED DESCRIPTION

The present invention generally relates to compositions and methods for the therapeutic induction of tolerance using recombinant cell surface antigens, in particular, soluble forms of cell surface proteins.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Certain ranges can be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided can be different from the actual publication dates which can need to be independently confirmed.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, particular materials and methods are described herein.

As used herein, the term “antigen” is intended to refer to a molecule capable of eliciting an immune response in a mammalian host, particularly a humoral immune response, i.e. characterized by the production of antigen-specific antibodies. Antigens of interest include therapeutic agents, e.g. polypeptides, and fragments thereof; autoantigens, e.g. self-polypeptides; transplantation antigens; and the like. In response to antigens, antibodies are produced in a variety of classes, subclasses and isotypes.

An “epitope” refers to the portion of the antigen bound by an antibody. Antigens may comprise multiple epitopes. Where the antigen is a protein, linear epitopes may range from about 5 to 20 amino acids in length. Antibodies may also recognize conformational determinants formed by non-contiguous residues on an antigen, and an epitope can therefore require a larger fragment of the antigen to be present for binding, e.g. a protein domain, or substantially all of a protein sequence. It will therefore be appreciated that a protein, which may be several hundred amino acids in length, can comprise a number of distinct epitopes.

For the purposes of the present invention, antigen specific “immune tolerance” is the absence of an immune response to a specific antigen in the setting of an otherwise substantially normal immune system. Tolerance is distinct from generalized immunosuppression, in which all, or all of a class such as B cell mediated immune responses are diminished.

Where the antigen of interest is an autoantigen, the induction of antigen specific immune tolerance may be sufficient to decrease the symptoms of the autoimmune disease in the patient, for example a patient may be sufficiently improved so as to maintain normal activities in the absence, or in the presence of reduced amounts, of general immunosuppressants, e.g. corticosteroids.

Another aspect of tolerance is the presence of an otherwise substantially normal immune system. The methods of the present invention are not directed to a general immunosuppression, and after the tolerizing regimen the immune response to antigens other than the antigen of interest are substantially normal.

“Treating” or “treatment” refers to either the prevention or the reduction or elimination of symptoms of the disease of interest, e.g., therapy. “Treating” or “treatment” can refer to the administration of a composition comprising a polypeptide of interest, e.g., Kell system antigens or antibodies raised against these antigens. Treatment can be prophylactic (to prevent or delay the onset of the disease, or to prevent the manifestation of clinical or subclinical symptoms thereof) or therapeutic suppression or alleviation of symptoms after the manifestation of the disease.

“Therapeutically-effective amount” refers to an amount of polypeptide, e.g., soluble cell surface antigen that is sufficient to prevent or to alleviate (e.g., mitigate, decrease, reduce) at least one of the symptoms associated with a disease or condition or to induce tolerance. It is not necessary that the administration of the composition eliminate the symptoms of the disease or condition, as long as the benefits of administration of compound outweigh the detriments. Likewise, the terms “treat” and “treating”, as used herein, are not intended to mean that the subject is necessarily cured of the disease or condition or that all clinical signs thereof are eliminated, only that some alleviation or improvement in the condition of the subject is effected by administration of the composition.

As used herein, the term “immune response” refers to the response of immune system cells to external or internal stimuli (e.g., antigen, cell surface receptors, cytokines, chemokines, and other cells) producing biochemical changes in the immune cells that result in immune cell migration, killing of target cells, phagocytosis, production of antibodies, other soluble effectors of the immune response, and the like.

Antigen-specific down modulation of immune responses has been achieved in two circumstances. The first is the use of RhoGam to prevent RhD—mothers from generating anti-D alloantibodies that can lead to fetal demise in utero. The second is the use of high dose factor VIII (fVIII) intravenously in order to shut off an anti-fVIII antibody inhibitor response in patients with hemophilia A who have become alloimmunized to replacement fVIII infusion. This high dose fVIII approach accomplishes antigen-specific down modulation of a pre-existing alloantibody.

The application of such approaches more widely is highly desirable and is the focus of this application. The disclosed subject matter provides additional methods for therapeutic induction of tolerance which is generally applicable to many clinically relevant conditions.

In one embodiment, the compositions and methods of the present invention can be applied to Hemolytic Disease of the Fetus and Newborn (HDFN), a well-described pathology in which a pregnant mother synthesizes alloantibodies against paternal antigens that are expressed on fetal red blood cells (RBCs). The mothers' alloantibodies cross the placenta and induce destruction of fetal RBCs and/or suppress fetal hematopoiesis. This results in fetal anemia that can lead to malformation, teratogenesis, and in some cases complete fetal demise leading to death (severe hydrops fetalis). This can happen in the context of a number of different RBC antigens, so long as the mother is negative and the father is positive. RhD antigen is far and away the most common offender in HDFN. However, rates of RhD induced HDFN have been significantly increased by the wide-spread use of Rh immune globulin, which prevents alloimmunization when given prophylactically. As a result of Rh immune globulin, rates of maternal antibodies to RhD are now very low. In contrast, no prophylaxis has been developed for the K antigen, which is the most immunogenic fetal antigen second to RhD. Accordingly, mothers who are lacking the K antigen can become alloimmunized to K and kill K positive fetuses in utero.

Given the success with tolerizing patients with anti-fVIII antibodies by giving large quantities of recombinant fVIII, it is a reasonable hypothesis that the same could be achieved by exposing patients with anti-K to large quantities of K antigen. However, unlike fVIII, which is naturally a soluble protein, K is a cell surface glycoprotein found on RBCs. Giving fVIII to a patient with anti-fVIII, results in neutralization and inactivation of fVIII without any ill effects to the patient. In stark contrast, transfusion of K+RBCs into a patient with anti-K can induce profound illness in some cases death) as a result of a hemolytic transfusion reaction (HTR). Accordingly, induction of tolerance in patients with anti-K through exposure to large quantities of K (in its natural state on RBCs) is not a viable approach due to the dangers of HTRs. To overcome the HTR based limitation of exposing patients with anti-K to K+RBCs, an aspect of the invention involves expressing the extracellular domain of K as a modified soluble Kell (sKEL1). In doing so, one can preserve the antigenic structure of the protein, allowing large doses of sKEL1 to be given to a patient with anti-K, without the risk of an HTR as the sKEL1 is no longer associated with a RBC.

As will be appreciated, the utility of this approach is not limited to K, but can be applied to any RBC antigen that can be expressed in the soluble form while maintaining the antigenic structure recognized by patient antibodies. For RBC alloantigens that are expressed on single membrane pass, GPI linked proteins, or linear peptides, this may be a readily accomplished (e.g. k, M, N, Fya, Fyb, Dombrock etc.). However, this approach may be more challenging with multipass transmembrane proteins that required proper insertion in a plasma membrane in order to maintain the tertiary structure responsible for the antigenic conformation (e.g., RhD, Diego, Kx, etc.).

In addition to expressing the soluble protein as itself, additional modifications can be made to the solubilized antigen to enhance its pharmacological properties. Such modifications may include:
1. Fusion to Fc domain of immunoglobulin to result in dimeric form and increased life-span upon infusion
2. Fusion to an immunomodulatory protein (other than Fc), including—but not limited to- FasL, CTLA4, or immunoregulatory cytokines (e.g. IL-10, TGF-beta., etc.)
3. Chemical modification with polyethyleneglycol (PEG)
4. Conjugation to a toxin (chemical or recombinant) to target naïve B cells that take up protein through receptor mediated endocytosis.

Furthermore, this approach is not limited to RBCs and would be applicable to any antigens that can be expressed in soluble form. Outside the context of HTR, additional potential diseases include platelet antigens responsible for Neonatal Alloimmune Immunethrombocytopenia (NAIT), and Autoimmune Hemolytic Anemia, Immune Thrombocytopenia (ITP). Additional applications may be found in other alloimmune or autoimmune diseases against receptors/antigens outside of hematological biology; including, but not limited to—Rasmussen's encephylitis, Hashimotos thyroiditis, Grave's disease, Myesthinea Gravis, etc.

### Polypeptides

The term “polypeptide” or “peptide” refers to a polymer of amino acids without regard to the length of the polymer; thus, peptides, oligopeptides, and proteins are included within the definition of polypeptide. This term also does not specify or exclude post-expression modifications of polypeptides, tier example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “isolated protein,” “isolated polypeptide,” or “isolated peptide” is a protein, polypeptide or peptide that by virtue of its origin or source of derivation (1) is not associated with naturally associated components that accompany it in its native state, (2) is free of other proteins from the same species, (3) is expressed by a cell from a different species, or (4) does not occur in nature. Thus, a peptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The terms “polypeptide”, “protein”, “peptide,” “antigen,” or “antibody” within the meaning of the present invention, includes variants, analogs, orthologs, homologs and derivatives, and fragments thereof that exhibit a biological activity, generally in the context of being able to induce an immune response in a subject, or bind an antigen in the case of an antibody.

The polypeptides of the invention include an amino acid sequence derived from Kell system antigens or fragments thereof, corresponding to the amino acid sequence of a naturally occurring protein or corresponding to variant protein, i.e., the amino acid sequence of the naturally occurring protein in which a small number of amino acids have been substituted, added, or deleted but which retains essentially the same immunological properties. In addition, such derived portion can be fluffier modified by amino acids, especially at the N- and C-terminal ends to allow the polypeptide or fragment to be conformationally constrained and/or to allow coupling to an immunogenic carrier after appropriate chemistry has been carried out. The polypeptides of the present invention encompass functionally active variant polypeptides derived from the amino acid sequence of Kell system antigens in which amino acids have been deleted, inserted, or substituted without essentially detracting from the immunological properties thereof, i.e. such functionally active variant polypeptides retain a substantial peptide biological activity.

In one embodiment, such functionally active variant polypeptides exhibit at least 60%, 65% 70%, 75%, 80% 81%, 82%, 83% 84%, 85%, 86% 87%, 88%, 89% 90%, 91%, 92%, 93%, 94%, 95%, 96%, 97%, 98%, or 99% identity to an amino acid sequence of the blood group antigens disclosed herein. Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides aligmnents and percent sequence identity of the regions of the best overlap between the queiy and search sequences (Pearson, Methods Enzymol. 183:63-98 (1990); Pearson, Methods Mol. Biol. 132:185-219 (2000)). An alternative algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997).

Functionally active variants comprise naturally occurring functionally active variants such as allelic variants and species variants and non-naturally occurring functionally active variants that can be produced by, for example, mutagenesis techniques or by direct synthesis.

A functionally active variant can exhibit, for example, at least 60%, 65%, 70%, 75%, 80%, 81%, 82%, 83%, 84%, 85%, 86%, 87%, 88%, 89% 90%, 91%, 92%, 93%, 94%, 95%, 96%, 97%, 98%, or 99% identity to an amino acid sequence of a Kell system or other antigen disclosed herein, and yet retain a biological activity. Where this comparison requires alignment, the sequences are aligned for maximum homology. The site of variation can occur anywhere in the sequence, as long as the biological activity is substantially similar to the Kell system or other antigens disclosed herein, e.g., ability to induce a tolerance reponse. Guidance concerning how to make phenotypically silent amino acid substitutions is provided in Bowie et al., Science, 247: 1306-1310 (1990), which teaches that there are two main strategies for studying the tolerance of an amino acid sequence to change. The first strategy exploits the tolerance of amino acid substitutions by natural selection during the process of evolution. By comparing amino acid sequences in different species, the amino acid positions which have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution can be modified while still maintaining specific immunogenic activity of the modified polypeptide.

The second strategy uses genetic engineering to introduce amino acid changes at specific positions of a cloned gene to identify regions critical for protein fimction. For example, site-directed mutagenesis or alanine-scanning mutagenesis can be used (Cunningham et al., Science, 244: 1081-1085 (1989)). The resulting variant polypeptides can then be tested for specific biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved.

Methods of introducing a mutation into amino acids of a protein are well known to those skilled in the art. See, e. g., Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989)).

Mutations can also be introduced using commercially available kits such as “QuikChange Site-Directed Mutagenesis Kit” (Stratagene) or directly by peptide synthesis. The generation of a functionally active variant to an peptide by replacing an amino acid which does not significantly influence the function of said peptide can be accomplished by one skilled in the art.

A type of amino acid substitution that may be made in the polypeptides of the invention is a conservative amino acid substitution. A “conservative amino acid substitution” is one in which an amino acid residue is substituted by another amino acid residue having a side chain R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See e.g. Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups may include: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

A functionally active variant can also be isolated using a hybridization technique. Briefly, DNA having a high homology to the whole or part of a nucleic acid sequence encoding the peptide, polypeptide or protein of interest, e.g. Kell system antigens, is used to prepare a functionally active peptide. Therefore, a polypeptide of the invention also includes entities which are functionally equivalent and which are encoded by a nucleic acid molecule which hybridizes with a nucleic acid encoding any one of the Kell system antigens or a complement thereof. One of skill in the art can easily determine nucleic acid sequences that encode peptides of the invention using readily available codon tables. As such, these nucleic acid sequences are not presented herein.

Nucleic acid molecules encoding a functionally active variant can also be isolated by a gene amplification method such as PCR using a portion of a nucleic acid molecule DNA encoding a peptide, polypeptide, protein, antigen, or antibody of interest, e.g. Kell system antigens, as the probe.

For the purpose of the present invention, it should be considered that several polypeptides or antigens of the invention may be used in combination. All types of possible combinations can be envisioned. The same sequence can be used in several copies on the same polypeptide molecule, or wherein peptides of different amino acid sequences are used on the same polypeptide molecule, the different peptides or copies can be directly fused to each other or spaced by appropriate linkers. As used herein the term “multimerized (poly)peptide” refers to both types of combination wherein polypeptides of either different or the same amino acid sequence are present on a single polypeptide molecule. From 2 to about 20 identical and/or different peptides can be thus present on a single multimerized polypeptide molecule.

In one embodiment of the invention, a peptide, polypeptide, protein, or antigen of the invention is derived from a natural source and isolated from a bacterial source. A peptide, polypeptide, protein, or antigen of the invention can thus be isolated from sources using standard protein purification techniques.

Alternatively, peptides, polypeptides and proteins of the invention can be synthesized chemically or produced using recombinant DNA techniques. For example, a peptide, polypeptide, or protein of the invention can be synthesized by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the filly automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989) and ‘Methods in Molecular Biology, Vol. 35: Peptide Synthesis Protocols (ed. M. W. Pennington and B. M. Dunn), chapter 7, pp 91-171 by D. Andreau et al.

Alternatively, a polynucleotide encoding a peptide, polypeptide or protein of the invention can be introduced into an expression vector that can be expressed in a suitable expression system using techniques well known in the art, followed by isolation or purification of the expressed peptide, polypeptide, or protein of interest. A variety of bacterial, yeast, plant, mammalian, and insect expression systems are available in the art and any such expression system can be used. Optionally, a polynucleotide encoding a peptide, polypeptide or protein of the invention can be translated in a cell-free translation system.

Nucleic acid sequences corresponding to Kell system antigens can also be used to design oligonucleotide probes and used to screen genomic or cDNA libraries for genes encoding other variants or from other species. The basic strategies for preparing oligonucleotide probes and DNA libraries, as well as their screening by nucleic acid hybridization, are well known to those of ordinary skill in the art. See, DNA Cloning: Vol. I, supra; Nucleic Acid Hybridization, supra; Oligonucleotide Synthesis, supra; Sambrook et al., supra. Once a clone from the screened library has been identified by positive hybridization, it can be confirmed by restriction enzyme analysis and DNA sequencing that the particular library insert contains a Kell system antigen gene, or a homolog thereof. The genes can then be further isolated using standard techniques and, if desired, PCR approaches or restriction enzymes employed to delete portions of the full-length sequence.

Alternatively, DNA sequences encoding the proteins of interest can be prepared synthetically rather than cloned. The DNA sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292: 756; Nambair et al. (1984) Science 223: 1299; Jay et al. (1984) J Biol. Chem. 259: 6311.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (grain-negative bacteria), pGV1106 (grain-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, Sambrook et al., supra; DNA Cloning; supra; B. Perbal, supra. The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence can or can not contain a signal peptide or leader sequence. Leader sequences can be removed by the host in post-translational processing. See, e.g., U.S. Pat. Nos. 4,431,739; 4,425,437; 4,338,397. Examples of vectors include pET32a(+) and pcDNA3002Neo.

Other regulatory sequences can also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements can also be present in the vector, for example, enhancer sequences.

The control sequences and other regulatory sequences can be ligated to the coding sequence prior to insertion into a vector, such as the cloning vectors described above. Alternatively, the coding sequence can be cloned directly into an expression vector which already contains the control sequences and an appropriate restriction site.

In some cases it can be necessary to modify the coding sequence so that it can be attached to the control sequences with the appropriate orientation; i.e., to maintain the proper reading frame. It can also be desirable to produce mutants or analogs of the protein. Mutants or analogs can be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, HEK293F cells, NSO-1 cells, as well as others. Similarly, bacterial hosts such as E. coli, Bacillus subtilis, and Streptococcus spp., will find use with the present expression constructs. Yeast hosts useful in the present invention include, but are not limited to, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, but are not limited to, Aedes aegypti, Autographa californica, Bombyx mori, Drosophila melanogaster, Spodoptera fmgiperda, and Trichoplusia ni.

Depending on the expression system and host selected, the proteins of the present invention are produced by culturing host cells transformed by an expression vector described above under conditions whereby the protein of interest is expressed. The protein is then isolated from the host cells and purified. The selection of the appropriate growth conditions and recovery methods are within the skill of the art.

Kell system antigen protein sequences can also be produced by chemical synthesis such as solid phase peptide synthesis, using known amino acid sequences or amino acid sequences derived from the DNA sequence of the genes of interest. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis. Chemical synthesis of peptides can be preferable if a small fragment of the antigen in question is capable of raising an immunological response in the subject of interest.

Polypeptides of the invention can also comprise those that arise as a result of the existence of multiple genes, alternative transcription events, alternative RNA splicing events, and alternative translational and postranslational events. A polypeptide can be expressed in systems, e.g. cultured cells, which result in substantially the same postranslational modifications present as when the peptide is expressed in a native cell, or in systems that result in the alteration or omission of postranslational modifications, e.g. glycosylation or cleavage, present when expressed in a native cell.

A peptide, polypeptide, protein, or antigen of the invention can be produced as a fusion protein that contains other distinct amino acid sequences that are not part of the Kell system antigen sequences disclosed herein, such as amino acid linkers or signal sequences or immunogenic carriers, as well as ligands useful in protein purification, such as glutathione-S-transferase, histidine tag, and staphylococcal protein A. More than one polypeptide of the invention can be present in a fusion protein. The heterologous polypeptide can be fused, for example, to the N-terminus or C-terminus of the peptide, polypeptide or protein of the invention. A peptide, polypeptide, protein, or antigen of the invention can also be produced as fusion proteins comprising homologous amino acid sequences.

### Blood Group Antigen Proteins

Any of a variety of soluble forms of cell surface proteins may be used in the practice of the present invention. In one embodiment, the proteins are soluble forms of blood group antigens, such as the Kell system antigens. Information on such antigens and, in particular, soluble forms are available in the art, for example, in Ridgwell et al., Transfusion Medicine, 17: 384-394 (2007).

Kell (CD238) is a clinically important human blood group antigen system comprising 28 antigens (Daniels et al., 2007, International Society of Blood Transfusion Committee on Terminology for Red Cell Surface Antigens: Cape Town report. Vox Sanguinis, 92, 250-253). The Kell antigens are carried by a single pass type II (cytoplasmic N-terminus) red blood cell membrane glycoprotein. The Kell glycoprotein is expressed in red cells and haematopoietic tissue (bone marrow and foetal liver) and to a lesser extent in other tissues, including brain, lymphoid organs, heart and skeletal muscle (Russo et al., 2000, Blood, 96, 340-346). The K/k (KEL1/KEL2) blood group antigen polymorphism is determined by a single nucleotide polymorphism (SNP) resulting in the presence of methionine (M) or threonine (T), respectively, at amino acid 193 of the extracellular C-terminal domain (Lee, 1997, Vox Sanguinis, 73, 1-11). The other most clinically significant antithetical antigens Kp^{a}/Kp^{b} (KEL3/KEL4) and Js^{a}/Js^{b} (KEL6/KEL7) are also the result of SNPs resulting in single amino acid changes in the extracellular domain (Lee, 1997, Vox Sanguinis, 73, 1-11).

Kell system antibodies are known to cause haemolytic transfusion reactions and haemolytic disease of the fetus and newborn (HDFN). Kell-related HDFN may be because of suppression of fetal erythropoiesis in addition to immune destruction of red blood cells as in most other cases of HDFN (Vaughan et al., 1998, New England Journal of Medicine, 338, 798-803; Daniels et al., 2003, Transfusion, 43, 115-116). Anti-K (KEL1) is the most commonly encountered immune red cell antibody outside the ABO and Rh systems, and other antigens of the Kell blood group system, e.g. k (KEL2), Kp^{a} (KEL3), Kp^{b} (KEL4), Js^{a} (KEL6) and Js^{b} (KEL7) are also capable of stimulating the production of haemolytic antibodies and causing HDFN (Daniels, 2002, Human Blood Groups (2nd edn). Blackwell, Oxford).

The Duffy (Fy, CD234) blood group antigens are carried by a type III membrane glycoprotein, which is predicted to span the membrane seven times with a glycosylated extracellular N-terminus and a cytoplasmic C-terminus. It is expressed in red blood cells, vascular endothelial cells and a wide range of other tissues including kidney, lung, liver, spleen, brain (Iwamoto et al., 1996, Blood, 87, 378-385) and colon (Chaudhuri et al., 1997, Blood, 89, 701-712). The Fy^{a}/Fy^{b} (FY1/FY2) blood group polymorphorism is determined by an SNP resulting in the presence of glycine (G) or aspartic acid (D), respectively, at amino acid 42 in the N-terminal extracellular domain (Iwamoto et al., 1995, Blood, 85, 622-626; Mallinson et al., 1995, British Journal of Haematology, 90, 823-82; Tournamille et al., 1995, Human Genetics, 95, 407-410). Duffy blood group system antibodies can cause haemolytic transfusion reactions (Boyland et al., 1982, Transfusion, 22, 402; Sosler et al., 1989, Transfusion, 29, 505-507) and HDFN (Vescio et al., 1987, Transfusion, 27, 366; Goodrick et al., 1997, Transfusion Medicine, 7, 301-304).

The Lutheran (Lu, B-CAM, CD239) blood group antigens are carried by two single-pass type I (cytoplasmic C-terminus) membrane glycoproteins, which differ in the length of their cytoplasmic domains [the B-CAM glycoprotein has a shorter C-terminal cytoplasmic tail than Lu (Campbell et al., 1994, Cancer Research, 54, 5761-5765)]. The Lu glycoprotein has five extracellular immunoglobulin-like domains and is a member of the immunoglobulin gene superfamily (IgSF) (Parsons et al., 1995, Proceedings of the National Academy of Science of the United States of America, 92, 5496-5500) and is expressed in red blood cells and a wide range of other tissues (Reid & Lomas-Francis, 2004, The Blood Group Antigens Factsbook (2nd edn). Academic Press, London). The Lu^{a}/Lu^{b} (LU1/LU2) blood group antigen polymorphism is determined by a SNP resulting in the presence of histidine (H) or arginine (R), respectively, at amino acid 77 of the first predicted N-terminal IgSF domain (El Nemer et al., 1997). Lutheran blood group system antibodies have been reported to be involved in mild delayed haemolytic transfusion reactions (Daniels, 2002, Human Blood Groups (2nd edn). Blackwell, Oxford) but are rarely involved in HDFN (Inderbitzen et al., 1982, Transfusion, 22, 542).

### Pharmaceutical Compositions

An aspect of the invention provides a composition comprising an effective tolerizing amount of an isolated Kell system antigen protein and a pharmaceutically acceptable carrier, wherein the composition is effective in a vertebrate subject to induce tolerance.

The compositions of the present invention can be prepared as injectables, either as liquid solutions or suspensions, or as solid forms which are suitable for solution or suspension in liquid vehicles prior to injection. The preparation can also be prepared in solid form, emulsified or the active ingredient encapsulated in liposome vehicles or other particulate carriers used for sustained delivery. For example, the material can be in the form of an oil emulsion, water in oil emulsion, water-in-oil-in-water emulsion, site-specific emulsion, long-residence emulsion, sticky emulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the active ingredient.

Polypeptides are formulated into compositions for delivery to a mammalian subject. The composition is administered alone, and/or mixed with a pharmaceutically acceptable vehicle or excipient. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants in the case of compositions, which enhance the effectiveness of the composition. Suitable adjuvants are described above. The compositions of the present invention can also include ancillary substances, such as pharmacological agents, cytokines, or other biological response modifiers.

Furthermore, the compositions including, for example, one or more Kell system antigens can be formulated into compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Actual methods of preparing such dosage forms are known, or will be apparent, to those skilled in the art. See, e.g., Remington's Pharmaceutical Sciences, Mack Publishing Company, Easton, Pa., current edition.

The composition is formulated to contain an effective amount of a protein, the exact amount being readily determined by one skilled in the art, wherein the amount depends on the animal to be treated. The composition or formulation to be administered will contain a quantity of one or more secreted proteins adequate to achieve the desired state in the subject being treated. For purposes of the present invention, a therapeutically effective amount of a composition comprising a protein, contains about 0.05 to 1500 μg protein, or about 10 to 1000 μg protein, or about 30 to 500 μg, or about 40 to 300 pg, or any integer between these values. For example, peptides of the invention can be administered to a subject at a dose of about 0.1 μg to about 200 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

Routes of administration include, but are not limited to, oral, topical, subcutaneous, intramuscular, intravenous, subcutaneous, intradermal, transdermal and subdermal. Depending on the route of administration, the volume per dose is about 0.001 to 10 ml, about 0.01 to 5 ml, or about 0.1 to 3 ml. Compositions can be administered in a single dose treatment or in multiple dose treatments (boosts) on a schedule and over a time period appropriate to the age, weight and condition of the subject, the particular formulation used, and the route of administration.

In some embodiments, a single dose of polypeptide or pharmaceutical composition according to the invention is administered. In other embodiments, multiple doses of a peptide or pharmaceutical composition according to the invention are administered. The frequency of administration can vary depending on any of a variety of factors, e.g., severity of the symptoms, whether the composition is used for prophylactic or curative purposes, etc. For example, in some embodiments, a peptide or pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a polypeptide according to the invention, e.g., the period of time over which a peptide is administered, can vary, depending on any of a variety of factors, e.g., patient response. etc. For example, a polypeptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Any suitable pharmaceutical delivery means can be employed to deliver the compositions to the vertebrate subject. For example, conventional needle syringes, spring or compressed gas (air) injectors (U.S. Pat. No. 1,605,763 to Smoot; U.S. Pat. No. 3,788,315 to Laurens; U.S. Pat. No. 3,853,125 to Clark et al.; U.S. Pat. No. 4,596,556 to Morrow et al.; and U.S. Pat. No. 5,062,830 to Dunlap), liquid jet injectors (U.S. Pat. No. 2,754,818 to Scherer; U.S. Pat. No. 3,330,276 to Gordon; and U.S. Pat. No. 4,518,385 to Lindcaner et al), and particle injectors (U.S. Pat. No. 5,149,655 to McCabe et al. and U.S. Pat. No. 5,204,253 to Sanford et al.) are all appropriate for delivery of the compositions.

If a jet injector is used, a single jet of the liquid composition is ejected under high pressure and velocity, e.g., 1200-1400 PSI, thereby creating an opening in the skin and penetrating to depths suitable for administration.

The compositions or polypeptides can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of the composition, peptides, or polypeptides are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions or polypeptides when administered orally, can be protected from digestion. This can be accomplished either by complexing the nucleic acid, polypeptide, or antibody with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacal. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

Compositions or polypeptides as aspects of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney, Nat. Biotechnol. 16: 153-157, 1998).

For inhalation, compositions or polypeptides as aspects of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Compositions or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide, of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186: 507-515, 1997.

In one aspect, the pharmaceutical formulations comprising compositions or polypeptides of the invention are incorporated in lipid monolayers or bilayers, e.g., liposomes, see, e.g., U.S. Pat. Nos. 6,110,490; 6,096,716; 5,283,185; 5,279,833. Aspects of the invention also provide formulations in which water soluble nucleic acids, peptides or polypeptides of the invention have been attached to the surface of the monolayer or bilayer. For example, peptides can be attached to hydrazide-PEG-(distcaroylphosphatidyl) ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the peptides and/or polypeptides of the invention are incorporated within micelles and/or liposomes (see, e.g., Sutures, J. Pharm. Pharmacol. 46: 23-28, 1994; Woodle, Pharm. Res. 9: 260-265, 1992). Liposomes and liposomal formulations can be prepared according to standard methods and arc also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.

In one aspect, the compositions are prepared with carriers that will protect the protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds can lie within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal model to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, generally of a labeled agent. Animal models useful in studies, e.g., preclinical protocols, are known in the art.

As defined herein, a therapeutically effective amount of the compositions, proteins or polypeptides (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day or per week for between about 1 to 10 weeks, for example, between 2 to 8 weeks, between about 3 to 7 weeks, or about 4, 5, or 6 weeks. In some instances the dosage can be required over several months or more. The skilled artisan will appreciate that certain factors can influence the dosage and timing required to effectively treat a subject, including, but not limited to the severity of the disease or disorder, previous treatments, the general health and/or age of the subject, and other diseases present. Moreover, treatment of a subject with a therapeutically effective amount of an agent such as a protein or polypeptide can include a single treatment or, can include a series of treatments.

The pharmaceutical compositions can be included in a container, pack, or dispenser together with instructions for administration.

Compounds as described herein can be used for the preparation of a medicament for use in any of the methods of treatment described herein.

The pharmaceutical compositions are generally formulated as sterile, substantially isotonic and in full compliance with all Good Manufacturing Practice (GMP) regulations of the U.S. Food and Drug Administration.

In various embodiments of the subject disclosure, the methods include performing blocking antibody-mediated RBC clearance by non-hemolytic antibodies, such as the antibodies provided herein. The methods can include producing and/or providing and/or administering the antibodies for the blocking. Various embodiments include administering an antigen and/or antibody, e.g., anti-Kell IgG1, and/or control phosphate buffered saline (PBS). The antibody and/or control can be administered in an amount of for example, 1 ug or less, 5 ug or less, 10 ug or less, 20 ug or less, or 50 ug or less. The antibody and/or control can be administered in an amount of for example, 1 ug or more, 5 ug or more, 10 ug or more, 20 ug or more, or 50 ug or more, such as 100 ug or more. The antibody and/or control can be administered in an amount ranging for example, from 1 ug to 100 ug, 5 ug to 50 ug, 5 ug to 15 ug, or 5 ug to 10 ug.

In various embodiments and as is shown, for example, in FIG, 1, antigens and/or antibodies as set forth herein, such as Anti-Kell IgG1, can cause a decrease in RBC survival of 70% or less as compared to a control. In various embodiments, antibodies as set forth herein, such as Anti-Kell IgG1, can cause a decrease in RBC survival of 90% or less, 80% or less, 75% or less, 70% or less, 65% or less, 60% or less, 50% or less as compared to a control. Antibodies as set forth herein, such as Anti-Kell IgG1, can cause a decrease in RBC survival of 90% or more, 80% or more, 75% or more, 70% or more, 65% or more, 60% or more, 50% or more as compared to a control. Antibodies as set forth herein, such as Anti-Kell IgG1, can also cause a decrease in RBC survival of 90% or more, 80% or more, 75% or more, 70% or more, 65% or more, 60% or more, 50% or more as compared to a control. Antibodies as set forth herein, such as Anti-Kell IgG1, can also cause a decrease in RBC survival ranging, for example, from 10% to 50%, 20% to 40%, or 25% to 35%.

Also, administration of an antigen and/or antibody as disclosed herein, e.g., a non-hemolytic antibodiy, such as the non-hemolytic form of anti-Kell (IgG3), can include reversing clearance by a hemolytic antibody, such as a hemolytic anti-Kell IgG1, to 50% or less, 40% or less, 30% or less, 20% or less, 15% or less, 10% or less, or 5% or less. Administration of an antibody as disclosed here, e.g., a non-hemolytic antibodiy, such as the non-hemolytic form of anti-Kell (IgG3), can include reversing clearance by a hemolytic antigen and/or antibody, such as a hemolytic anti-Kell IgG1, to 50% or more, 40% or more, 30% or more, 20% or more, 15% or more, 10% or more, or 5% or more. Such administration can also include reversing clearance by a hemolytic antibody a percent range, for example, 1% to 25%, 5% to 20%, 5% to 15%, or 5% to 10% or 10% to 15%.

In some embodiments, the method sinclude reducing a transfusion recipient's reisk of developing an hemolytic transfusion reaction (HTR). In some versions the methods include performing a blood transfusion on a subject. In some versions, the methods include not performing a transfustion when an amount of antigen and/or antibody as set forth herein are not first administered.

### Treatment Regimens: Pharmacokinetics

The pharmaceutical composition aspects of the invention can be administered in a variety of unit dosage forms depending upon the method of administration. Dosages for typical compositions, peptides, or polypeptides are well known to those of skill in the art. Such dosages are typically advisory in nature and are adjusted depending on the particular therapeutic context or patient tolerance. The amount of peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient at risk for a disease or condition in an amount sufficient to at least partially arrest or prevent the condition or a disease and/or its complications. For example, in one aspect, a composition comprising a soluble peptide pharmaceutical composition dosage for intravenous (IV) administration would be about 0.01 ing/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

In therapeutic applications, compositions are administered to a patient in an amount sufficient to at least partially arrest or prevent a condition or a disease and/or its complications and/or to induce tolerance. For example, in one aspect, a composition comprising a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

### Methods of Treatment

Also described herein are both prophylactic and therapeutic methods of treating a subject at risk of (or susceptible to) a disorder or a method of preventing or treating a disease or condition by administering a composition of the invention.

### Prophylactic Methods

An aspect of the invention relates to methods for preventing or treating in a subject a disease or condition by administering a composition comprising an effective tolerizing amount of protein and pharmaceutically acceptable carrier, wherein the composition is effective in a vertebrate subject to reduce or eliminate a disease or condition. Subjects at risk for a disorder or undesirable symptoms that are caused or contributed to by a particular disease or condition can be identified by, for example, any of a combination of diagnostic or prognostic assays as described herein or are known in the art. Administration of the agent as a prophylactic agent can occur prior to the manifestation of symptoms, such that the symptoms are prevented, delayed, or diminished compared to symptoms in the absence of the agent.

### Therapeutic Methods

An aspect of the invention relates to methods for preventing or treating in a subject a a disease or condition by administering a composition comprising an effective tolerizing; amount of a protein and a pharmaceutically acceptable carrier, wherein the composition is effective in a vertebrate subject to reduce or eliminate the disease or condition.

The following examples of specific aspects for carrying out the present invention are offered for illustrative purposes only, and are not intended to limit the scope of the present invention in any way.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted. for. Unless indicated otherwise, parts are parts by weight, molecular weight is weight average molecular weight, temperature is in degrees Centigrade, and pressure is at or near atmospheric.

### Example 1

### Expression and Purification of Soluble Cell Surface Blood Group Proteins

A number of methods available in the art to express and purify recombinant proteins may be used in the practice of the present invention. Among these include the methods disclosed in Ridgwell et al., Transfusion Medicine, 17: 384-394 (2007).

For example, the complementary DNA (cDNA) encoding the extracellular C-terminal domain of the Kell glycoprotein (amino acid residues 68-732) encoding k, Kp^{b} and Js^{b} antigens can be amplified by polymerase chain reaction (PCR) from a full-length cDNA clone. The PCR product can be cut with appropriate restrictions enzymes and cloned in frame into an appropriate vector, which contains three adjacent FLAG epitopes (Asp-Tyr-Lys-Xaa-Xaa-Asp) upstream of the cloning site. The K variant can be produced by site-directed mutagenesis of the above cDNA clone.

The cDNA encoding the extracellular N-terminal domain (amino acid residues 1-66, N-terminal sequence MASSGYV) of the Duffy glycoprotein encoding Fy^{a} or Fy^{b} antigens can be amplified by PCR from full-length cDNA clones and cloned in frame into an appropriate vector, which contains the FLAG epitopes downstream of the cloning site.

The cDNA encoding the extracellular IgSF domains (amino acid residues 1-515) of the Lu^{b} glycoprotein can be cloned in an eukaryotic cell expression vector pIg, which contains the genomic Fc region of human IgG1 downstream of the cloning site (Simmons, 1993, Cellular Interactions in Development: A Practical Approach. IRL Press, Oxford; Parsons et al., 2001, Proceedings of the National Academy of Science of the United States of America, 92, 5496-5500). The Lu^{a} variant can be produced by PCR mutagenesis of the Lu^{b} cDNA clone.

Stably transfected COS-7 cells secreting recombinant FLAG fusion proteins can be produced by electroporation of COS-7 cells with the expression plasmids above. Secreted recombinant proteins can be isolated and purified from cell culture supernatant using anti-FLAG-M2 agarose affinity chromatography with 3XFLAG peptide elution. Secreted recombinant Lu-IgFc fusion proteins can be produced in transiently transfected COS-7 cells and can be isolated and purified from cell culture supernatant using protein A sepharose.

### Example 2

### Blocking of Antibody-Mediated RBC Clearance by Non-Hemolytic Antibodies

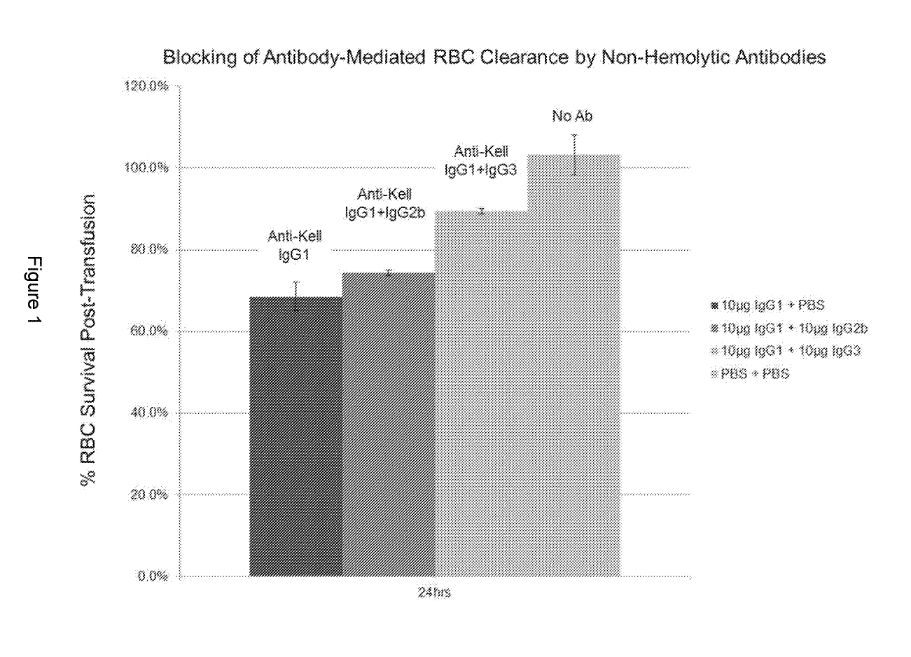

Experiments were conducted directed to blocking antibody-mediated RBC clearance by non-hemolytic antibodies. In the experiments performed, recipient mice received either 10 ug of hemolytic anti-Kell IgG1 or control PBS. As is shown, for example, in FIG. 1. Anti-Kell IgG1 caused a decrease in RBC survival to 70% compared to control. However, addition of the non-hemolytic form of anti-Kell (IgG3) reversed clearance by the hemolytic anti-Kell IgG1 to only 10%, These studies do not reflect anti-Kell further engineered to eliminate any residual hemolysis of the IgG3 form. These findings demonstrate, for example, that protectabodies work in an in vivo pre-clinical model.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed:

1. A method for inducing immune tolerance against a cell surface antigen in a subject comprising administering to the subject an effective amount of a soluble form of the cell surface antigen sufficient to induce tolerance to said antigen in the subject.

2. The method of claim 1, wherein the subject has previously been sensitized.

3. The method of claim 1, wherein the subject has developed an antibody as a result of previous exposure to the antigen.

4. The method of claim 1, wherein the cell surface antigen is a red blood cell antigen.

5. The method of claim 4, wherein the red blood cell antigen is an antigen within the Kell system comprising an allelic variant of the extracellular domain of the Kell glycoprotein.

6. The method of claim 5, wherein said allelic variants are selected from the group consisting of K, k, Kpb, Kpa, Jsb and Jsa antigens.

7. The method of claim 4, wherein the red blood cell antigen is a variant selected from within the Duffy, Kidd, or Lewis systems.

8. The method of claim 1, wherein the cell surface antigen is a platelet antigen.

9. The method of claim 8, wherein the platelet antigen is selected from Human Platelet Antigens 1-16 (HPA 1-16).

10. The method of claim 1, wherein the antigen is a neutrophil antigen (HNA antigens).

11. The method of claim 1, wherein the antigen is an HLA variant of MHC I or MHC II.

12. The method of claim 1, wherein said administering is performed prior or subsequent to the onset of a disease or condition that is caused by said cell surface antigen.

13. The method of claim 12, wherein the disease or condition is hemolytic transfusion reaction (HTR).

14. The method of claim 12, wherein the disease or condition is Neonatal Alloimmune Immunethrombocytopenia (NAIT), Autoimmune Hemolytic Anemia, or Immune Thrombocytopenia (ITP).

15. The method of claim 12, wherein the disease or condition is Rasmussen's encephylitis, Hashimotos thyroiditis, Grave's Disease, Pemphigus Vulgaris, Stiffman syndrome, or Myasthenia gravis.

16. The method of claim 1, wherein the soluble form of the cell surface antigen is fused to the Fe domain of an immunoglobulin.

17. The method of claim 1, wherein the soluble form of the cell surface antigen is fused to an immunomodulatory protein.

18. The method of claim 17, wherein the immunomodulatory protein is FasL, CTLA4, or an immunoregulatory cytokine.

19. The method of claim 18, wherein the immunoregulatory cytokine is IL-10 or TGF-beta.

20. The method of claim 1, wherein the soluble antigen is modified with PEG.

21. The method of claim via the soluble antigen is conjugated to a toxin.
